(19) United States
(12) Patent Application Publication
(10) Pub. No.: US 2009/0198102 A1
(43) Pub. Date: Aug. 6, 2009

(54) Rectum Examination Device And Rectum Examination Set

(76) Inventors: Tien-Sheng Chen, Taipei City (TW); Hui-Bih Yuan, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/325,301

(22) Filed: Dec. 1, 2008

(30) Foreign Application Priority Data
Jan. 31, 2008 (TW) 097103792

(51) Int. Cl. A61B 1/00; A61B 1/04

(52) U.S. Cl. 600/114; 600/109

(57) ABSTRACT

This invention relates to a rectum examination device capable of connecting to an endoscope with an image capture element. The rectum examination device comprises a check pipe comprising a large open end and a small open end; the small open end can enter the rectum of a human body, and the caliber of the small open end is smaller than the caliber of the large open end; and a container installed in the inner side of the check pipe, the container being capable of containing and separating the image capture element, the container comprising an open portion and a closed portion.

### BACKGROUND OF THE INVENTION

1. Field of the Invention

The present invention relates to a rectum examination device and a rectum examination set; more particularly, the present invention relates to a rectum examination device and a rectum examination set capable of containing and separating an image capture element.

2. Description of the Related Art

In order to diagnose symptoms of rectal disease, such as tumors, piles, and ulcers, a proctoscope is used for diagnosis. During the traditional rectal proctoscope diagnosis, a check pipe is first inserted the rectum of a human body to facilitate the insertion of an endoscope. The check pipe also can prevent injury to anal tissue. The endoscope captures images of the rectum with an image capture element installed at its front end. The doctor can thus determine whether the rectal tissue is abnormal according to the image transmitted from the endoscope, and the image is stored in a memory unit as a record of the examination.

However, the image capture element of the endoscope is not isolated during the traditional proctoscope diagnosis. Therefore, during the diagnosis, the image capture element may make contact with the patient or being contaminated by rectal excretions or gas. If disinfection is not conducted after each diagnosis, cross infection may result. However, it is time consuming to conduct disinfection after each diagnosis, and there may be still doubt about whether the disinfection is complete. In addition, since the traditional anal endoscope is not integrated with the check pipe, the doctor needs to use both hands to hold the endoscope and the check pipe during the diagnosis, which is inconvenient.

### SUMMARY OF THE INVENTION

It is therefore an object of the present invention to provide a rectum examination device capable of containing and separating an image capture element.

It is another object of the present invention to provide a rectum examination set capable of containing and separating an image capture element.

To achieve the first object mentioned above, the rectum examination device of the present invention comprises a check pipe comprising a large open end and a small open end; the small open end can enter the rectum of a human body, and the caliber of the small open end is smaller than the caliber of the large open end; and a container installed in the inner side of the check pipe, the container being capable of containing and separating the image capture element, the container comprising an open portion and a closed portion.

To achieve another object mentioned above, the rectum examination set of the present invention comprises an endoscope and a rectum examination device. The endoscope comprises a main body accommodating a control module and a slender part connected to the main body; the slender part comprises an image capture element electrically connected to the control module to allow control by the control module. The rectum examination device comprises a check pipe comprising a large open end and a small open end, the small open end being able to enter a* rectum of a human body, and the caliber of the small open end is smaller than the caliber of the large open end; and a container installed in the inner side of the check pipe, the container being capable of containing and separating the image capture element, the container comprising an open portion and a closed portion.

Other objects, advantages, and novel features of the present invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 is an illustration of one embodiment of a rectum examination device of the present invention.

FIG. 2 is a cross-section view of the rectum examination device of the present invention.

FIG. 3 is an illustration of one embodiment of an endoscope of the present invention.

FIG. 4 is an illustration of one embodiment of a rectum examination set of the present invention.

FIG. 4A is a cross section view of the rectum examination set of FIG. 4 along A-A.

FIG. 5 is an illustration of one embodiment of a rectum examination set of the present invention.

### DETAILED DESCRIPTION OF THE PREFERRED EMBODIMENT

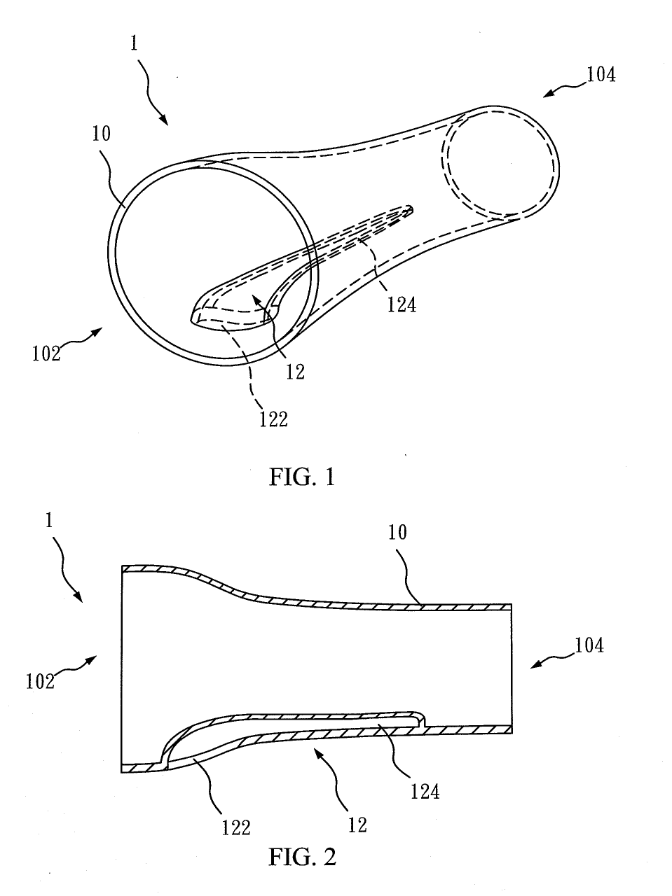

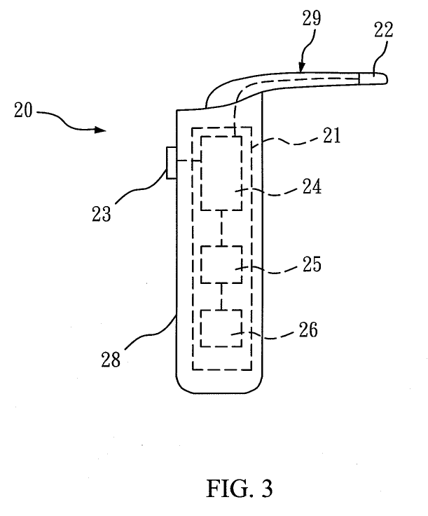

Please refer to FIG. 1 and FIG. 2. FIG. 1 is an illustration of one embodiment of a rectum examination device of the present invention. FIG. 2 is a cross-section view of the rectum examination device 1. The rectum examination device 1 of the present invention comprises a check pipe 10 and a container 12. The check pipe 10 is capable of connecting to an endoscope. The check pipe 10 comprises a large open end 102 and a small open end 104; the small open end 104 can enter the rectum of a human body, and the caliber of the small open end 104 is smaller than the caliber of the large open end 102.

The container 12 is installed in the inner side of the check pipe 10. The container 12 is capable of containing and separating an image capture element of the endoscope, the container 12 comprising an open portion 122 and a closed portion 124. The closed portion 124 is in a slender form and extends from the large open end 102 to the small open end 104, such that the image capture element of the endoscope can capture clear images of deeper locations. In the present embodiment, the container 12 is transparent, and the open portion 122 of the container 12 is separate from the large open end 102 of the check pipe 10; that is to say, the open portion 122 is recessed inside the large open end 102.

Particularly, the open portion 122 is not limited to the above location; for example, the open portion 122 of the container 12 could also be not separate from the large open end 102 of the check pipe 10; that is to say, the open portion 122 could be aligned with the large open end 102.

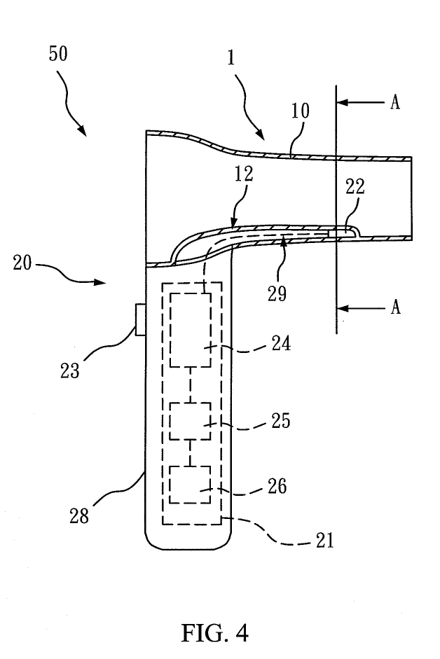

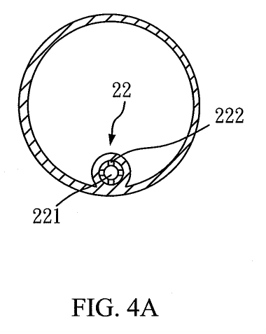

Please refer to FIG. 3, FIG. 4, and FIG. 4A together. FIG. 3 is an illustration of one embodiment of an endoscope of the present invention. FIG. 4 is an illustration of one embodiment of a rectum examination set of the present invention, and FIG. 4A is a cross section view along A-A of FIG. 4. The rectum examination set 50 comprises a rectum examination device 1 and an endoscope 20. The endoscope 20 comprises a main body 28 and a slender part 29, wherein the slender part 29 is connected to the main body. The rectum examination device 1 and the endoscope 20 can be combined together by containing the slender part 29 in the container 12.

The slender part 29 comprises an image capture element 22. In the present embodiment, the image capture element 22 is set at one end of the slender part 29. The image capture element 22 can generate corresponding image data of the captured image for analysis by the user. The main body 28 accommodates a control module 21. The control module 21 is electrically connected to the image capture element 22 for controlling the image capture element 22.

The slender part 29 comprises a wire inside for electrically connecting the image capture element 22 and the control module 21. The slender part 29 can be made of soft material in order to move the image capture element 22 to the front end of the closed portion 124 easily.

The whole image capture element 22 can be contained and separated inside the container 12, therefore preventing contact between the image capture element 22 and the human body; that is to say, the endoscope 20 can be reused without a disinfection process and will not cause cross infection. The image capture element 22 comprises an image sensing unit 221 and a light unit 222. The image sensing unit 221 is for sensing images, and can be a CCD (Charge Coupled Device), a CMOS (Complementary metal oxide semiconductor), or other type of image sensing unit.

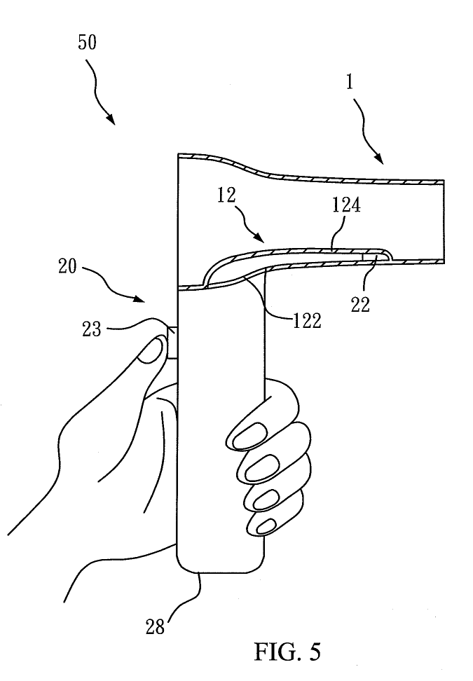

In the present embodiment, the control module 21 comprises a battery 24, a wireless transmitter 25, and a memory unit 26 wherein the battery 24 provides electrical power to the endoscope 20. The wireless transmitter 25 can wirelessly transmit image data generated by the image capture element 22 to a remote computer (not shown in FIG) for display by and storage in the remote computer. Therefore, there is no need to use a physical wire to transmit image data to a monitor, thus providing greater convenience. The memory unit 26 can store image data generated by the image capture element 22. Please refer to FIG. 5, and refer to FIG. 4 and FIG. 4A as well. FIG. 5 is an illustration of one embodiment of a rectum examination set of the present invention. As shown in FIG. 5, the user combines the rectum examination device 1 and the endoscope 20 by putting the image capture element 22 through the open portion to the end of the closed portion with assistance of the slender part 29, in order to capture a clearer image. The user can operate a switch 23 to switch on/off the image capture element 22 according to need. Because the image capture element 22 is isolated inside the container 12 during the diagnosis process, the image capture element 22 will not be contaminated by contact with the human body. Therefore, after the diagnosis is finished, the user only needs to discard the rectum examination device 1, and the image capture element 22 can be easily detached from the container 12 and put into another rectum examination device for the next examination.

In contrast to the prior art, the rectum examination device 1 of the present invention is capable of containing and separating the image capture element 22 inside the container 12 to prevent the image capture element 22 from being contaminated, thus preventing cross infection and increasing the safety of a rectal examination. In addition, the rectum examination device 1 and the endoscope 20 of the present invention can be combined together to provide more convenience in use.

Although the present invention has been explained in relation to its preferred embodiments, it is to be understood that many other possible modifications and variations can be made without departing from the spirit and scope of the invention as hereinafter claimed.

What is claimed is:

1. A rectum examination device capable of connecting to an endoscope with an image capture element, the rectum examination device comprising:
a check pipe comprising a large open end and a small open end; the small open end can enter an rectum of a human body, and the caliber of the small open end is smaller than the caliber of the large open end; and
a container installed in the inner side of the check pipe, the container being capable of containing and separating the image capture element, the container comprising an open portion and a closed portion.

2. The rectum examination device as claimed in claim 1, wherein the open portion of the container is substantially aligned with the large open end of the check pipe.

3. The rectum examination device as claimed in claim 1, wherein the open portion of the container is recessed inside the large open end of the check pipe.

4. The rectum examination device as claimed in claim 1, wherein the closed portion of the container is in a slender form.

5. The rectum examination device as claimed in claim 4, wherein the open portion of the container is recessed inside the large open end of the check pipe.

6. A rectum examination set comprising:
an endoscope comprising:
a main body accommodating a control module; and
a slender part connected to the main body, the slender part comprising:
an image capture element electrically connected to the control module for being controlled by the control module; and
a rectum examination device comprising:
a check pipe comprising a large open end and a small open end; the small open end can enter a rectum of a human body, and the caliber of the small open end is smaller than the caliber of the large open end; and
a container installed in inner side of the check pipe, the container being capable of containing and separating the image capture element, the container comprising an open portion and a closed portion.

7. The rectum examination set as claimed in claim 6, wherein the image capture element comprises:
an image sensing unit for sensing images.

8. The rectum examination set as claimed in claim 7, wherein the image sensing unit is a CCD (Charge Coupled Device) or a CMOS (Complementary metal oxide semiconductor).

9. The rectum examination set as claimed in claim 7, wherein the image capture element further comprises a light unit for emitting light.

10. The rectum examination set as claimed in claim 9, wherein the light unit comprises a light emitting diode.

11. The rectum examination set as claimed in claim 6, wherein the control module comprises a battery.

12. The rectum examination set as claimed in claim 6, wherein the control module further comprises a wireless transmitter for transmitting image data generated by the image capture element.

13. The rectum examination set as claimed in claim 6, wherein the control module further comprises a memory unit for storing image data generated by the image capture element.

14. The rectum examination device as claimed in claim 6, wherein the open portion of the container is substantially aligned with the large open end of the check pipe.

15. The rectum examination device as claimed in claim 6, wherein the open portion of the container is recessed inside the large open end of the check pipe.

16. The rectum examination device as claimed in claim 6, wherein the closed portion of the container is in a slender form.

17. The rectum examination device as claimed in claim 16, wherein the open portion of the container is recessed inside the large open end of the check pipe.

18. The rectum examination set as claimed in claim 6, wherein the control module comprises a battery, a wireless transmitter, and a memory unit, wherein the wireless transmitter is for transmitting image data generated by the image capture element, and the memory unit is for storing image data generated by the image capture element.
